(19) United States
(12) Patent Application Publication
(10) Pub. No.: US 2018/0193528 A1
(43) Pub. Date: Jul. 12, 2018

(54) PRINTABLE MORPHOGENETIC PHASE-SPECIFIC CHITOSAN-CALCIUM-POLYPHOSPHATE SCAFFOLD FOR BONE REPAIR

(71) Applicant: Werner Ernst Ludwig Georg MÜLLER, Mainz (DE)

(72) Inventors: Werner Ernst Ludwig Georg MÜLLER, Mainz (DE); Heinrich-Christoph Wilhelm Friedrich SCHRÖDER, Wiesbaden (DE); Xiaohong WANG, Mainz (DE)

(21) Appl. No.: 15/322,229

(22) PCT Filed: Jul. 24, 2015
(86) PCT No.: PCT/EP2015/066979

§ 371 Date: Dec. 27, 2016

(30) Foreign Application Priority Data
Jul. 24, 2014 (GB) 1413154.4

(51) Int. Cl. A61L 27/46; A61L 27/20; A61L 27/52; A61L 27/56; A61L 27/58; C12N 5/077

(52) U.S. Cl. CPC A61L 27/46; A61L 27/20; A61L 27/52; C08L 5/04; A61L 27/58; C12N 5/0654; A61L 2430/02; A61L 27/56

(57) ABSTRACT

This invention concerns a formula for the synthesis of a printable hybrid material, formed of carboxymethyl chitosan (CMC) and polyphosphate (polyP). Both polymers are linked together by calcium ions. The inventive CMC-polyP material, in combination with alginate, is biocompatible, biodegradable and useful for three-dimensional (3D) printing and 3D cell printing (bioprinting). The CMC-polyP scaffold, hardened by exposure to calcium ions, is morphogenetically active and can be used in bone N tissue engineering, as a bio mimetic 3-phase scaffold that mimics and induces essential phases in bone repair, including blood clot formation and platelet degranulation (release of growth factors and cytokines) (Phase 1: initiation phase), calcium carbonate bioseed formation (Phase 2: nucleation) and expression/activation of bone alkaline phosphatase (Phase 3: hydroxyapatite-biomineral.

This invention concerns a formula for the synthesis of a printable hybrid material, formed of carboxymethyl chitosan (CMC) and polyphosphate (polyP). Both polymers are linked together by calcium ions. The inventive CMC-polyP material [composition of CMC with polyP], in combination with alginate, is biocompatible, biodegradable and useful for three-dimensional (3D) printing and 3D cell printing (bioprinting). The CMC-polyP scaffold, hardened by exposure to calcium ions, is morphogenetically active and can be used in bone tissue engineering, as a biomimetic 3-phase scaffold that mimics and induces essential phases in bone repair, including blood clot formation and platelet degranulation (release of growth factors and cytokines) (Phase 1: initiation phase), calcium carbonate bioseed formation (Phase 2: nucleation) and expression/activation of bone alkaline phosphatase (Phase 3: hydroxyapatite-biomineral formation).

### BACKGROUND OF INVENTION

Biological bone substitutes must meet the requirements to be highly porous and to offer a microenvironment for regenerative cells, e.g. support cell attachment, proliferation, differentiation, and, by that, initiate and maintain neo-tissue genesis. To fabricate a three-dimensional (3D) scaffold for tissue engineering various metals have been exploited. In spite of their advantageous mechanical properties metals have the disadvantage not to be biodegradable. In parallel, inorganic/ceramic materials, e.g. hydroxyapatite (HA) or calcium phosphates, have been developed that display the desired osteoconductivity, but are difficult to produce in a highly porous structure and are brittle. Finally, biomimetic artificially designed scaffolds that mimic the structures of living systems provide the feature of the physiological extracellular matrix to recruit cells in the implanted biomaterial. In this context, chitosan derived from chitin, is of particular interest.

Chitosan

Chitosan is a polysaccharide derived from chitin that is randomly built by 3-(1-4)-linked D-glucosamine and N-acetyl-D-glucosamine units. Chitosan shows suitable properties for tissue engineering purposes. This polymer is biocompatible and biodegradable, and can be used both for 3D-scaffolds, as gels and tissue-like units, and for 2D-scaffolds, as films and fibers (Croisier F, Jérôme C. Chitosan-based biomaterials for tissue engineering. Europ Polymer J 2013; 49:780-792). Chitosan has been used for space-filling implants. However, this natural polymer has to be processed with morphogenetically active components, e.g. silica, to become a suitable matrix for bone regeneration (Shirosaki Y, Tsuru K, Hayakawa S, Osaka A, Lopes M A, Santos J D, Costa M A, Fernandes M H. Physical, chemical and in vitro biological profile of chitosan hybrid membrane as a function of organosiloxane concentration. Acta Biomater 2009; 5:346-355).

Essential Phases During Bone Repair

Bone repair is a process that can be divided in multiple phases that could be affected by “intelligent”, phase-specific scaffold materials.

Phase 1—Blood Coagulation and Effect of polyP:

Bone repair is initiated by blood coagulation at the site of the bone defect. The dense granules of human platelets contain substantial amounts of polyP, with chain lengths of 70-75 (Ruiz F A, Lea C R, Oldfield E, Docampo R. Human platelet dense granules contain polyphosphate and are similar to acidocalcisomes of bacteria and unicellular eukaryotes. J Biol Chem 2004; 279:44250-44257) or 60-100 phosphate units (Müller F, Mutch N J, Schenk W A, Smith S A, Esterl L, Spronk H M, Schmidbauer S, Gahl W A, Morrissey J H, Renné T. Platelet polyphosphates are proinflammatory and procoagulant mediators in vivo. Cell 2009; 139:1143-1156), which is released upon platelet activation (Smith S A, Mutch N J, Baskar D, Rohloff P, Docampo R, Morrissey J H. Polyphosphate modulates blood coagulation and fibrinolysis. Proc Natl Acad Sci USA 2006; 103:903-908). PolyP secreted by platelets acts as a hemostatic regulator; it is a procoagulant agent that accelerates blood clotting by promoting the activation of factor V and activation of the contact pathway (Smith S A, Morrissey J H. Polyphosphate as a general procoagulant agent. J Thromb Haemost 2008; 6:1750-1756). On the other hand, polyP delays clot lysis by enhancing the thrombin-activatable fibrinolysis inhibitor (Smith S A, Mutch N J, Baskar D, RohloffP, Docampo R, Morrissey J H. Polyphosphate modulates blood coagulation and fibrinolysis. Proc Natl Acad Sci USA 2006; 103:903-908). A series of growth factors are found in blood clot, including fibroblast growth factor-2 (FGF-2), epidermal growth factor (EGF), platelet-derived growth factor (PDGF), insulin-like growth factor (IGF), transforming growth factor beta (TGF-b) and vascular endothelial growth factors (VEGF). These factors can enhance different phases of osteogenesis. For example, the proliferation of osteoblastic progenitor cells is stimulated by PDGF, EGF and FGF-2.

Phase 2—Calcium Carbonate Bioseed Formation:

It could be demonstrated that biocalcite (CaCO₃) fulfils crucial roles during initiation of bone HA formation. It has been demonstrated that CaCO₃ deposits function as bio-seeds for Ca-phosphate precipitation onto bone forming cells (Müller W E G, Schröder H C, Schlossmacher U, Grebenjuk V A, Ushijima H, Wang X H. Induction of carbonic anhydrase in SaOS-2 cells, exposed to bicarbonate and consequences for calcium phosphate crystal formation. Biomaterials 2013; 34:8671-8680). In particular, the carbonic anhydrase not only facilitates bicarbonate/calcium carbonate biomineral formation but also acts in concert with the polyP/pyrophosphate-degrading bone alkaline phosphatase (tissue-nonspecific ALP), through the initial formation of Ca-carbonate deposits.

Phase 3—Hydroxyapatite Deposition:

The initially formed Ca-carbonate deposits are subsequently transformed into Ca-phosphate/HA minerals by the ALP, opening the development of new strategies for therapeutic intervention of bone diseases, such as the development of morphogenetically active implant materials (Wang X H, Schröder H C, Müller W E G. Enzyme-based biosilica and biocalcite: biomaterials for the future in regenerative medicine. Trends Biotechnol 2014, in press; doi: 10.1016/j.tibtech.2014.05.004).

Polyphosphate (polyP)

Previously the inventors published the role of polyP during HA deposition on bone cells. PolyP is a linear polymer occurring in nature of two up to hundreds of phosphate residues (Schröder H C, Müller W E G, eds. Inorganic Polyphosphates—Biochemistry, Biology, Biotechnology. Prog Mol Subcell Biol 1999; 23:45-81). PolyP can be synthesized both chemically and enzymatically (Kulaev I S, Vagabov V, Kulakovskaya T. The Biochemistry of Inorganic Polyphosphates. New York: John Wiley & Sons Inc; 2004).

PolyP is enzymatically formed by polyphosphate kinases and enzymatically degraded by exo- and endopolyphosphatases (reviewed in: Schröder H C, Lorenz B, Kurz L, Müller W E G. Inorganic polyP in eukaryotes: enzymes, metabolism and function. In Schröder H C, Müller W E G, eds, Inorganic Polyphosphates—Biochemistry, Biology, Biotechnology. Prog Mol Subcell Biol 1999; 23:45-81).

Several enzymes that degrade polyP are known (e.g., Lorenz B, Müller W E G, Kulaev I S, Schröder H C. Purification and characterization of an exopolyphosphatase activity from Saccharomyces cerevisiae. J Biol Chem 1994; 269:22198-22204), among them the bone ALP (Lorenz B, Schröder H C. Mammalian intestinal alkaline phosphatase acts as highly active exopolyphosphatase. Biochim Biophys Acta 2001; 1547:254-261).

The bone ALP (tissue-nonspecific ALP) is an exopolyphosphatase that degrades polyP by a processive mechanism to monomeric phosphate (Lorenz B, Schröder H C. Mammalian intestinal alkaline phosphatase acts as highly active exopolyphosphatase. Biochim Biophys Acta 2001; 1547:254-261).

PolyP is present in bone tissue (Leyhausen G, Lorenz B, Zhu H, Geurtsen W, Bohnensack R, Müller W E G, Schröder H C. Inorganic polyphosphate in human osteoblast-like cells. J Bone Mineral Res 1998; 13:803-812; Schröder H C, Kurz L, Müller W E G, Lorenz B. Polyphosphate in bone. Biochemistry (Moscow) 2000; 65:296-303) and in platelets (Smith S A, Mutch N J, Baskar D, Rohloff P, Docampo R, Morrissey J H. Polyphosphate modulates blood coagulation and fibrinolysis. Proc Natl Acad Sci USA 2006; 103:903-908).

PolyP is morphogenetically active after complex formation with Ca²⁺ ions (polyP.Ca²⁺ complex or polyP.Ca²⁺-salt); the polyP.Ca²⁺-complex
induces the expression and enhances the activity of the bone ALP (tissue non-specific ALP) (Müller W E G, Wang X H, Diehl-Seifert B, Kropf K, Schloßmacher U, Lieberwirth I, Glasser G, Wiens M, Schröder H C. Inorganic polymeric phosphate/polyphosphate is an inducer of alkaline phosphatase and a modulator of intracellular Ca²′ level in osteoblasts (SaOS-2 cells) in vitro. Acta Biomater 2011; 7:2661-2671);
increases the intracellular Ca² level in osteoblasts (Müller W E G, Wang X H, Diehl-Seifert B, Kropf K, Schloßmacher U, Lieberwirth I, Glasser G, Wiens M, Schröder H C. Inorganic polymeric phosphate/polyphosphate is an inducer of alkaline phosphatase and a modulator of intracellular Ca²⁺ level in osteoblasts (SaOS-2 cells) in vitro. Acta Biomater 2011; 7:2661-2671);
enhances the expression of the gene encoding BMP-2 in bone (HA)-forming cells (Wang X H, Schröder H C, Diehl-Seifert B, Kropf K, Schloßmacher U, Wiens M, Müller W E G. Dual effect of inorganic polymeric phosphate/polyphosphate on osteoblasts and osteoclasts in vitro. J Tissue Eng Regen Med 2013; 7: 767-776);
induces HA formation (Wang X H, Schröder H C, Diehl-Seifert B, Kropf K, Schloßmacher U, Wiens M, Müller W E G. Dual effect of inorganic polymeric phosphate/polyphosphate on osteoblasts and osteoclasts in vitro. J Tissue Eng Regen Med 2013; 7: 767-776); and
inhibits the differentiation of osteoclasts (Wang X H, Schröder H C, Diehl-Seifert B, Kropf K, Schloßmacher U, Wiens M, Müller W E G. Dual effect of inorganic polymeric phosphate/polyphosphate on osteoblasts and osteoclasts in vitro. J Tissue Eng Regen Med 2013; 7: 767-776).

The following patent applications concerning polyP or polyP.Ca²⁺-complex are relevant:

GB1406840.7. Morphogenetically active hydrogel for bioprinting of bioartificial tissue. Inventors: Müller W E G, Schröder H C, Wang X H.
GB1403899.6. Synergistic composition comprising quercetin and polyphosphate for treatment of bone disorders. Inventors: Müller W E G, Schröder H C, Wang X H.
GB1319416.2. Modulator of bone mineralization based on a combination of polyphosphate/carbonate and carbonic anhydrase activators. Inventors: Müller W E G, Schröder H C, Wang X H.

In this invention, a complex of polyP with chitosan is described that can be used as a biomimetic material for bone tissue engineering and repair that features controlled morphology and displays morphogenetic activity.

Chitosan cannot form a complex with polyP at physiological conditions. Therefore the inventors derivatized chitosan to N,O-carboxymethyl chitosan (N,O-CMC) using state-of-the-art procedures (Chen S C, Wu Y C, Mi F L, Lin Y H, Yu L C, Sung H W. A novel pH-sensitive hydrogel composed of N,O-carboxymethyl chitosan and alginate cross-linked by genipin for protein drug delivery. J Control Release 2004; 96:285-300; Anitha A, Divya Rani V V, Krishna R, Sreeja V, Selvamurugan N, Nair S V, Tamura H, Jayakumar R. Synthesis, characterization, cytotoxicity and antibacterial studies of chitosan, O-carboxymethyl and N,O-carboxymethyl chitosan nanoparticles. Carbohydrate Polymers 2009; 78:672-677). The ampholytic character of N,O-CMC provides this polymer with an ample of applications (Mourya V. K, Inamdar N N and Tiwari A. Carboxymethyl chitosan and its applications. Adv Mat Lett 2010; 1:11-33).

Biosilica

Biosilica is a naturally occurring polymer that is formed enzymatically from ortho-silicate by the enzyme silicatein (Müller W E G, Schröder H C, Burghard Z, Pisignano D, Wang X H. Silicateins: a paradigm shift in bioinorganic chemistry. Enzymatic synthesis of inorganic polymeric silica. Chem Eur J 2013; 19:5790-5804).

Biosilica has an inductive anabolic effect on bone-forming cells; it increases the expression of BMP-2 and causes a shift of the osteoprotegerin: RANKL ratio, resulting in an inhibition of differentiation of pre-osteoclasts into mature osteoclasts (reviewed in: Wang X H, Schröder H C, Wiens M, Ushijima H, Müller W E G. Bio-silica and bio-polyphosphate: applications in biomedicine (bone formation). Curr Opin Biotechnol 2012; 23:570-578).

The following patents or patent applications concerning biosilica are relevant:

EP1320624; U.S. Pat. No. 7,169,589B2; DE10037270; CN01813484.X; NZ523474; AU2001289713. Silicatein-mediated synthesis of amorphous silicates and siloxanes and their uses. Inventors: Müller W E G, Lorenz A, Krasko A, Schröder H C.
U.S. Pat. No. 6,670,438B1. Methods, compositions, and biomimetic catalysts for in vitro synthesis of silica, polysilsequioxane, polysiloxane, and polymetallo-oxanes. Inventors: Morse D E, Stucky G D, Deming, T D, Cha J, Shimizu K, Zhou Y.
DE10246186. In vitro and in vivo degradation or synthesis of silicon dioxide and silicones, useful e.g. for treating silicosis or to prepare prosthetic materials, using a new silicase enzyme. Inventors: Müller W E G, Krasko A, Schröder H C.
EP1546319. Abbau und Modifizierung von Silicaten und Siliconen durch Silicase und Verwendung des reversiblen Enzyms. Inventors: Müller W E G, Krasko A, Schröder H C.
EP1740707; U.S. Ser. No. 11/579,019; DE10352433.9; CA2565118; JP2007509991. Enzym-und Template-gesteuerte Synthese von Silica aus nicht-organischen Siliciumverbindungcn sowie Aminosilanen und Silazanen und Verwendung. Inventors: Schwertner H, Müller W E G, Schröder H C.
EP09005849.6. Use of silintaphin for the structure-directed fabrication of (nano)composite materials in medicine and (nano)technology. Inventors: Wiens M, Müller W E G, Schröder HC, Wang X.
DE102004021229.5; EP2005004738; U.S. Ser. No. 11/579,020; JP2007509992; CA2565121. Enzymatic method for producing bioactive, osteoblast-stimulating surfaces and use thereof. Inventors: Müller. W E G, Schwertner H, Schröder H C.
U.S. 60/839,601; EP 2007007363. Biosilica-adhesive protein nano-composite materials: synthesis and application in dentistry. Inventors: Müller W E G, Schröder H C, Geurtsen W K.
PCT/US2009/005302. Compositions, oral care products and methods of making and using the same. Inventors: Miller J, Höfer H, Geurtsen W, Lücker P, Wiens M, Schröder H C, Müller W E G.
GB14059943. Osteogenic material to be used for treatment of bone defects. Inventors: Müller W E G, Schröder H C, Wang X H.

Bioglass

Bioglasses (bioactive glasses) are printable hard bone-imitating scaffold materials. The present state-of-the-art is reviewed in (Hench L L. Bioactive materials for gene control. In Hench L L, Jones J R, Fenn M B, eds, New Materials and Technologies for Healthcare. Singapore: World Scientific, pp 25-48, 2011; Jones J R. Review of bioactive glass: from Hench to hybrids. Acta Biomater 2013; 9:4457-4486).

The following patent application concerning bioglasses is relevant:

GB1408402.4. 3D cell printing of bioglass-containing scaffolds by combination with cell-containing morphogenically active alginate/gelatin hydrogels. Inventors: Müller W E G, Schröder H C, Wang X H.

Biomimetic materials gain increasing importance in tissue engineering since they may represent regenerative alternatives to harvested tissues for transplantation. Among the three-dimensional templates, mimicking the physiological extracellular matrix, chitosan and N,O-carboxymethyl chitosan (N,O-CMC) are widely used. In order to provide these polymers with a biological function additional components have to be added.

The inventors developed a formula for the preparation of a bioprintable material, composed of alginate, N,O-CMC and Na-polyP. After printing of this material to custom-designed/fabricated layers and implants, the structures are exposed to Ca²⁺ in order to harden them. During Ca²⁺ exposure the Na⁺ cations in the polyP are exchanged by Ca²⁺ allowing the bridging of polyP to N,O-CMC and rendering the composite material particularly stable without loosing the biological activity of polyP.

The inventors describe the formulation and fabrication of N,O-CMC-based polyP hybrid material. The two polymers are linked together via Ca²⁺ bridges in a stable way and provide a porous structure. The material can be printed to implants filling μCT analyzed lesions. Since the material retains its biological morphogenetic function, initiating biomineralization onto SaOS-2 bone-like cells, and accelerates blood clotting, N,O-CMC-polyP represents a promising new material applicable in tissue engineering of bone defects.

### DETAILED DESCRIPTION OF THE INVENTION

This invention is related to the formula for the synthesis of a new hybrid material, formed of N,O-CMC and polyphosphate (polyP), a natural polymer. Both polymers are linked together via Ca²⁺ bridges. Those N,O-CMC-polyP materials retain their morphology in culture medium and are especially useful for bioprinting. The N,O-CMC-polyP printed layers and tissue units also retain their biological function, to induce bone cells to biomineralization, and to accelerate the clotting process of human blood and, in turn, represent a promising new material useful for tissue engineering purposes.

The inventive scaffold consists of carboxymethyl chitosan, polyphosphate (sodium salt), and alginate (sodium salt), and is fabricated by 3D printing (bioprinting) of the resulting hydrogel and subsequent hardening by exposure to calcium ions.

The carboxymethyl chitosan can be formed by carboxymethylation of the amino groups of chitosan (N-carboxymethyl chitosan) or the hydroxy groups of chitosan (O-carboxymethyl chitosan) or both (N,O-carboxymethyl chitosan).

The non-carboxymethylated amino groups and/or the non-carboxymethylated hydroxy groups of the carboxymethyl chitosan can be acetylated or partially acetylated.

The novel biomimetic 3-phase scaffold according to this invention has the following properties; it is:
biocompatible,
biodegradable, and
printable.

This scaffold mimics three essential phases in bone repair; it affects the following 3 target sites which are active during 3 phases of bone repair:

Phase 1:

Clot formation associated with the release of growth factors/cytokines from platelets (initiation phase)

Phase 2:

Calcium carbonate bioseed formation by providing nucleation centers at the carboxymethal chitosan backbone (seed phase)

Phase 3:

Expression/activation of bone alkaline phosphatase (Hydroxvapatite-biomineral formation phase)

The finding that the inventive material, containing polyP linked via Ca²⁺ to the N,O-CMC polymer, has a significantly stronger effect than the published chitosan+polyP-PEC complex (Mi F L, Shyu S S, Wong T B, Jang S F, Lee S T, Lu K T. Chitosan-polyelectrolyte complexation for the preparation of gel beads and controlled release of anticancer drug. H. Effect of pH-dependent ionic crosslinking or interpolymer complex using tripolyphosphate or polyphosphate as reagent. J Appl Polymer Sci 1999; 74:1093-1107; Ong S Y, Wu J, Moochhala S M, Tan M H, Lu J. Development of a chitosan-based wound dressing with improved hemostatic and antimicrobial properties. Biomaterials 2008; 29:4323-4332) was unexpected, for the following reasons: (i) From published results, it is likely that the effect of polyP on clot formation does not require complex or salt formation of the polyP with calcium ions; in the contrary, the effect of polyP on coagulation has even been observed after preincubation of plasma before addition of calcium ions (Smith S A, Mutch N J, Baskar D, Rohloff P, Docampo R, Morrissey J H. Polyphosphate modulates blood coagulation and fibrinolysis. Proc Natl Acad Sci USA 2006; 103:903-908). (ii) Moreover, it is surprising that polyP, in the form of a calcium salt, is biologically active even after complex formation with the N,O-CMC polymer.

In addition, it was surprising that the inventive formulation (i) is printable and (ii) the printed meshwork shows sufficient stability after 3D printing—in contrast to the predictions based on the properties of the individual materials alone.

The alginate can be supplemented with gelatin or another collagen-derived product.

In addition, the inventors show that the inventive alginate-CMC-polyP hydrogel can be supplemented with silica or biosilica that stimulates bone-forming cells to mineralize and to express morphogenetically active cytokines, e.g. BMP-2. The polymeric silica or biosilica can be enzymatically formed by silicatein.

The technology according to this invention can be applied for the fabrication of cell-containing scaffold/implants, in particular scaffolds containing bone-forming cells or bone-dissolving cells or a mixture of these cells, whereby the cells are suspended in the alginate hydrogel and the resulting cell-containing alginate-CMC-polyP hydrogel is subjected to 3D printing (bioprinting) and subsequent hardening by exposure to calcium ions.

The hydrogel can be simultaneously printed, using a 3D printing technique, with a suspension of bioglass (bioactive glass) (nano)particles that can be composed of SiO₂:CaO:P₂O₅ or SiO₂:Na₂O:CaO:P₂O₅ of various molar ratios, for example SiO₂:CaO:P₂O₅ of a molar ratio (mol. %) of 55:40:5 or SiO₂:Na₂O:CaO:P₂O₅ of a molar ratio (mol. %) of 46.1:24.4:26.9:2.6 (45S5 Bioglass®).

The average chain lengths of the polyP molecules can be in the range 10 to up to 100 phosphate units. Optimal results were obtained with polyP molecules with an average chain length of about 40 phosphate units.

The polymeric silicic acid that can be added as an additional component can be formed by an enzyme or protein involved in biosilica (amorphous, hydrated silicon oxide) metabolism, such as silicatein or a silicatein fusion protein. The silicatein polypeptide or a silicatein fusion protein can be produced using a prokaryotic or eukaryotic expression system, or can be produced synthetically.

The silicatein or silicatein fusion protein can be present together with a suitable substrate (silica precursor) such as water glass, orthosilicic acid, orthosilicates, monoalkoxysilanetriols, dialkoxysilanediols, trialkoxysilanols, tetraalkoxysilanes, alkyl-silanetriols, alkyl-silanediols, alkyl-monoalkoxysilanediols, alkyl-monoalkoxysilanols, alkyl-dialkoxysilanols, or alkyl-trialkoxysilanes.

A further aspect of the invention concerns a 3D-bioprinted scaffold obtained by one of the methods described above, used as a bone implant or a material forming part of such implant.

The bone implant material can be produced for the treatment of a bone defect in the form of a customized implant by 3D printing, 3D cell printing (bioprinting) or another rapid prototyping procedure.

The invention will now be described further in the following preferred examples, nevertheless, without being limited thereto. For the purposes of the present invention, all references as cited herein are incorporated by reference in their entireties. In the Figures,

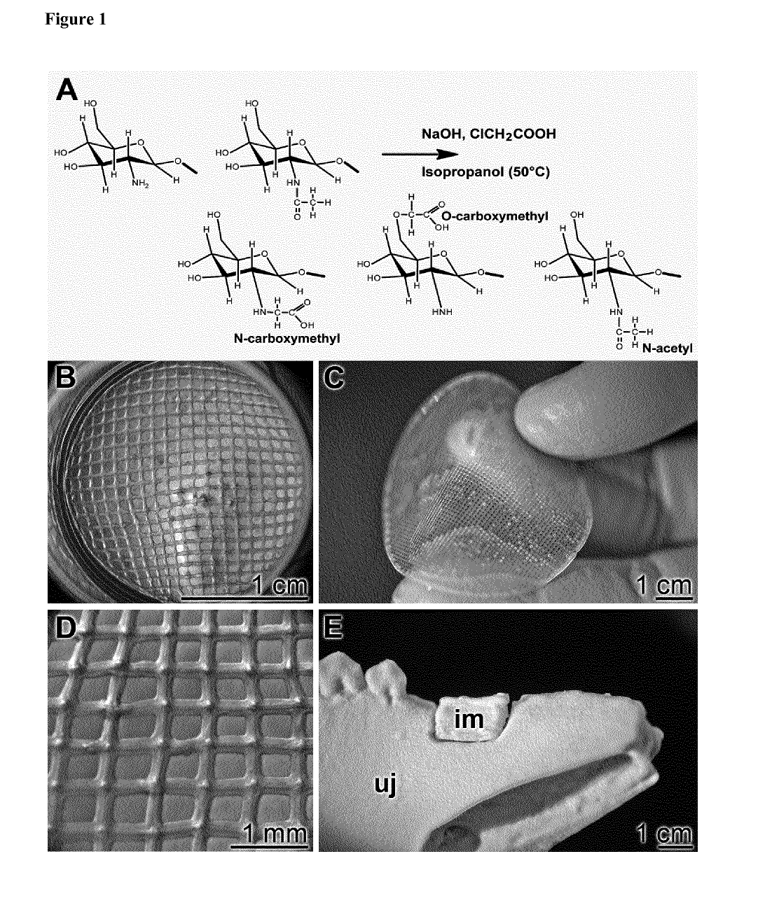

FIG. 1 shows the formation of N,O-CMC-polyP membranes and tissue units. (A) Chitosan, characterized by the D-glucosamine (deacetylated) and N-acetyl-D-glucosamine (acetylated) units, is converted into N,O-CMC by partial carboxymethylation of the polymer. (B to E) Mats of two (B and C) to six layers (D) were bioprinted. (E) Printing of a N,O-CMC-polyP tissue-like unit, an implant (im), formed according to the lesion in a pig underjaw (uj).

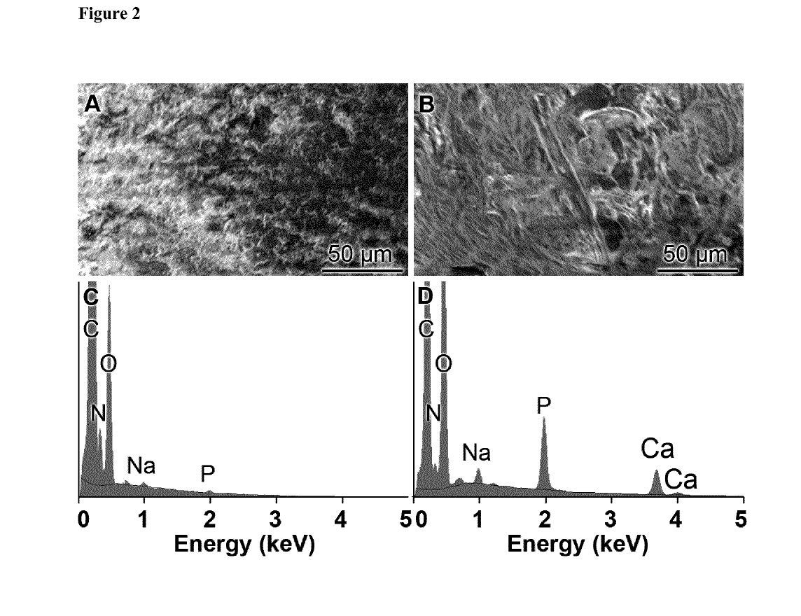

FIG. 2 shows the EDX analysis of membranes formed of N,O-CMC. The membranes were prepared in the absence of polyP (A and C) or in the presence of polyP (B and D). only in the EDX spectrum of the N,O-CMC-polyP membranes the signals for phosphorous and calcium show up.

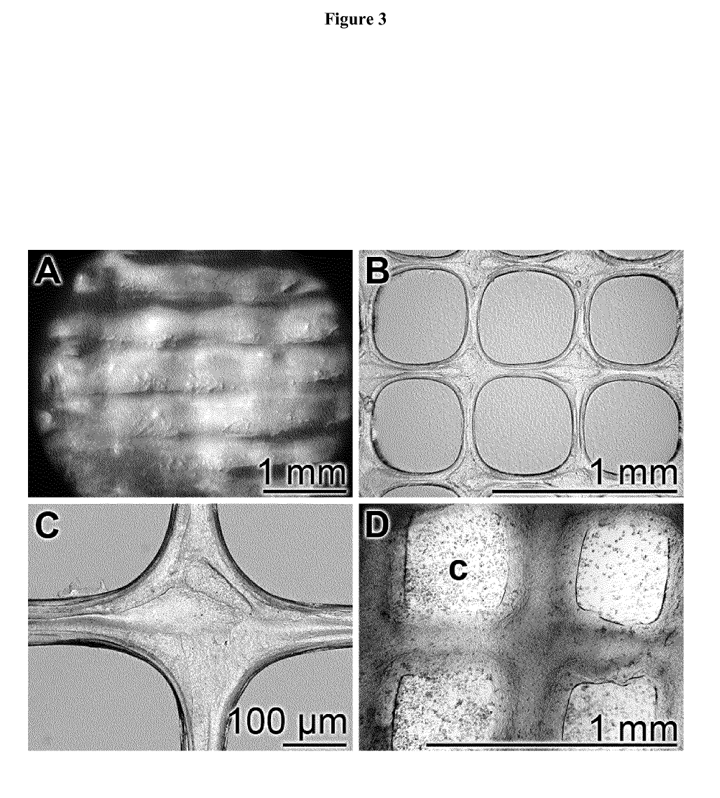

FIG. 3 shows the integrity and stability of the N,O-CMC-polyP meshwork. In contrast to the scaffold meshes build from (A) N,O-CMC, not containing polyP, which fuse, the N,O-CMC-polyP meshes remain intact even if submersed in culture medium. (B and C) Freshly prepared N,O-CMC-polyP meshwork. It is seen that only at the crossing points a fusion of the printed cylinders is seen; a continuous crossing point is formed. (D) Even after an incubation period of the N,O-CMC-polyP mesh in culture medium for 5 d, the cylinders remains separated and allow the cells (c) to proliferate in the open space.

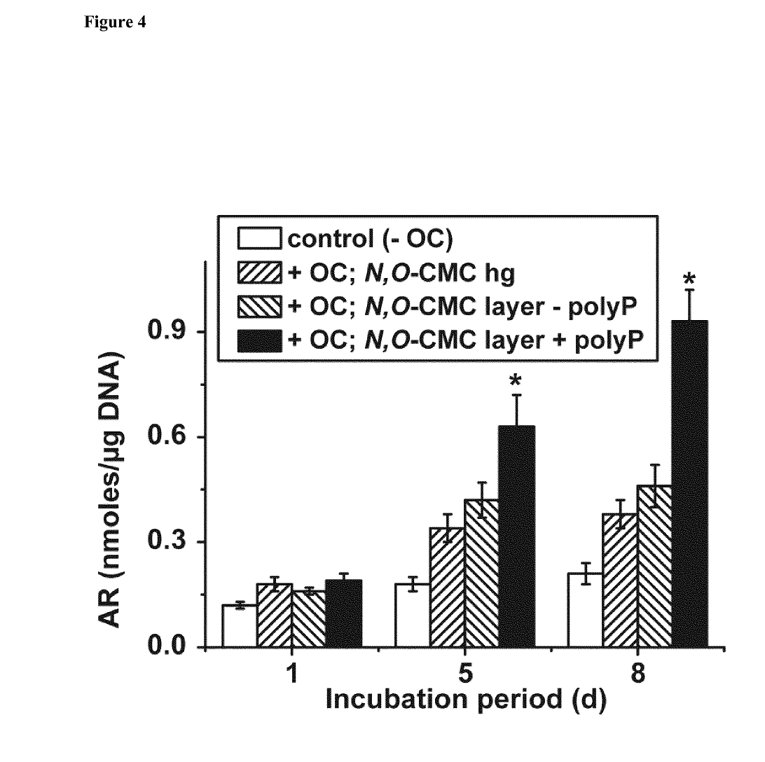

FIG. 4 shows the potency of SaOS-2 cells to mineralize on chitosan matrices. The SaOS-2 cells were grown in the absence (control; —OC) or presence of the OC. In the latter assays the cells were cultured on the previously published N,O-CMC hydrogel (N,O-CMC hg), or the N,O-CMC layers, in the absence (N,O-CMC−polyP) or presence of polyP (N,O-CMC+polyP). The extent of biomineralization (Alizarin Red S [AR]) is correlated with the DNA content in the assays. Values represent the means (±SD) from 10 separate experiments each. The N,O-CMC-polyP matrix significantly increases the mineralization; *P<0.01.

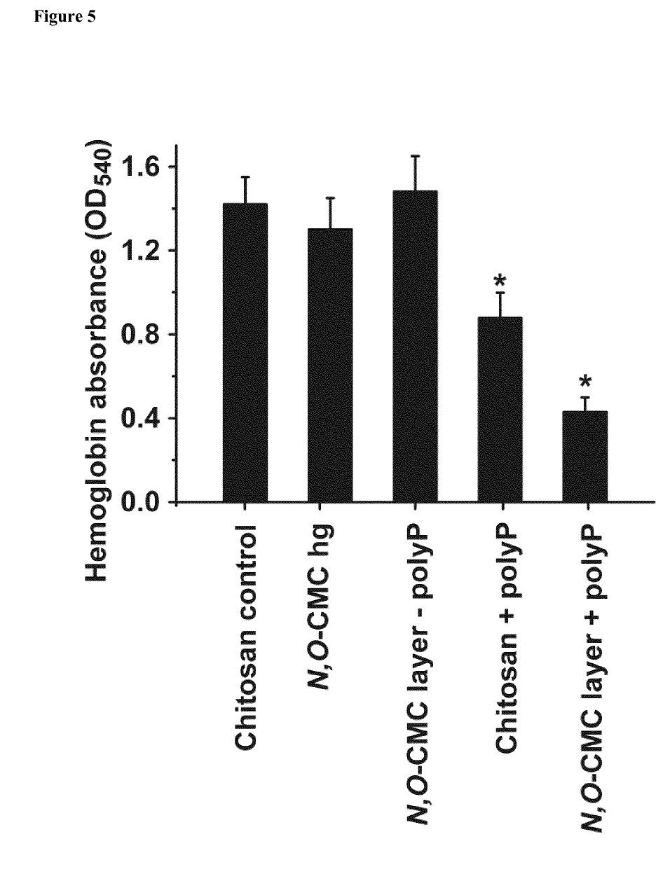

FIG. 5 shows the effect of chitosan polyP complex (“Chitosan+polyP”), N,O-CMC hydrogel (“N,O-CMC hg”) and N,O-CMC layers minus polyP (“N,O-CMC layer−polyP”) and plus polyp (“N,O-CMC layer+polyP”) on blood clotting rates. In the control non-processed chitosan had been added. The absorbance of hemoglobin from lysed noncoagulated erythrocytes was determined. The significance was compared to the chitosan control; *p<0.05.

### EXAMPLES

In the following examples, only the inventive method described, using N,O-CMC-polyP membranes, layers and tissue-like-blocks. Nevertheless, the method according to this invention can also be applied using O-carboxmethyl chitosan (O-CMC) and N-carboxymethyl chitosan (N-CMC) and polyP molecules with chain lengths other than 40 phosphate units, and the person of skill will be able to adjust the method as described accordingly.

Preparation of N,O-CMC-Polyphosphate Membranes, Layers and Tissue-Like-Blocks

As described under “Methods” the N,O-CMC-polyP was prepared. The N,O-CMC was mixed with Na-polyP; then the two polymers were linked together via Ca²⁺ ionic bridges. The membranes, layers or tissue-like-blocks were analyzed for the presence of phosphorus by EDX spectroscopy. As an example the EDX spectra from membranes, prepared without Na-polyP and with Na-polyP are given (FIGS. 2A and B). The surfaces of the membranes were analyzed. The spectra show that the membranes that had been formed in the presence of Na-polyP, and then linked via Ca²⁺ to the N,O-CMC polymer showed the signals for phosphorous and calcium (FIG. 2D), while those signals are absent in the membranes formed in the absence of polyP (FIG. 2C).

Printing of N,O-CMC-Polyphosphate Layers and Tissue-Like-Blocks

Using the setting as described above, two or six layers were printed and used for the in vitro assays. In FIG. 1 B and C the two-layer mats for the in vitro studies are shown. The mesh size of the cylinders was ≈0.5×0.5 mm.

The thickness of the layers can be increased by increasing the numbers of layers. A six-layer pad is shown in FIG. 2D. Increasing further the layering, tissue-like blocks are formed (FIG. 1E). Here the inventors printed a cranial defect in a pig underjaw, after having analyzed the lesion by CT.

N,O-CMC-polyP Layers Assayed in Cell Culture

The two layer printed scaffolds were used for the cell culture experiments. If a sample from a N,O-CMC layer, lacking any polyP, has been printed the cylinders fuse in the culture medium (FIG. 3A). In contrast, if this material to be printed is supplemented with polyP, the N,O-CMC-polyP, then the cylinders remain separated (FIGS. 1B and D). Even more, the crossing cylinders fuse only at the intimate, initial crossing points, under formation of continuous attachment mesh between the two layers (FIGS. 3B and C). The distinct intersections between the printed cylinders leave room for the infiltration of cells (FIG. 3D). Even after a five days' incubation period the meshwork remain intact (FIG. 3D).

Mechanical Properties of the N,O-CMC-polyP Material

The hardness of the N,O-CMC-polyP scaffold was measured with an indenter device and using a cantilever on the top of a glass ferrule. Scaffold samples of 6 layers with a thickness of 2 mm were measured. The samples that had been obtained immediately after printing were submersed into saline and tested for the hardness, using the reduced Young's modulus [RedYM] as a parameter. If those samples, N,O-CMC-polyP were analyzed a mechanical RedYM stiffness 935±128 kPa was determined (n=10); in contrast, the samples from the scaffold lacking the polyP measured only 27±3 kPa. In comparison, and using the same settings the trabecular bone from a rabbit tissue was found to have a modulus of 2,300 kPa. Submersing the N,O-CMC-polyP scaffold samples in simulated body fluid (Kokubo T. Bioactive glass ceramics: properties and applications. Biomaterials 1991; 12:155-163) the RedYM stiffness changed not significantly during a 3 weeks' period; the values are around 900±115 kPa; only after 6 weeks a significant reduction to 686±102 kPa is measured.

Mineralization of SaOS-2 Cells on N,O-CMC Matrices

The matrices as prepared here, N,O-CMC without and with polyP, as well as (in comparison) the chitosan preparation, termed N,O-CMC hydrogel, published earlier (Chen X G, Park H J. Chemical characteristics of O-carboxymethyl chitosans related to the preparation conditions. Carbohydrate Polymers 2003; 53:355-359; Luo Y, Teng Z, Wang X, Wang Q. Development of carboxymethyl chitosan hydrogel beads in alcohol-aqueous binary solvent for nutrient delivery applications. Food Hydrocolloids 2013a; 31: 332-339), were tested for their potency to induce in SaOS-2 cells biomineralization. The cells were transferred after an initial incubation period for 3 d in medium/FCS supplemented with the OC.

As shown in FIG. 4, N,O-CMC-polyP, which contains polymeric polyP bound to the N,O-CMC, caused a significantly higher induction of the mineralization of the cells (0.93±0.09 nmoles of Alizarin Red S bound to the cells [based on g of DNA] at day 8) than N,O-CMC matrices, lacking polyP. This holds true for the published N,O-CMC hydrogel matrix (0.38.0.04 nmoles/μg), and the matrix prepared here (0.46±0.06 nmoles/μg). In the absence of any chitosan matrix the extent of mineralization was 0.38±0.07 nmoles/μg (not shown in FIG. 4). In the absence of the OC the level of mineralization was low with ≈0.20±0.03 nmoles/μg.

Effect of N,O-CMC-polyP on Kinetics of Blood Clotting

PolyP is known to promote clot formation (Smith S A, Mutch N J, Baskar D, Rohloff P, Docampo R, Morrissey J H. Polyphosphate modulates blood coagulation and fibrinolysis. Proc Natl Acad Sci USA 2006; 103:903-908) and also to reverse anticoagulation and bleeding episodes in patients with hemophilia (Smith S A, Morrissey J H. Polyphosphate as a general procoagulant agent. J Thromb Haemost 2008; 6:1750-1756). In turn the inventors measured here the effect of the different matrices on the clotting time of human blood in vitro. The determinations were performed with whole blood contacted with similar amounts of matrices. After a contact period of 10 min the remaining erythrocytes in suspension were measured on the basis of their hemoglobin content (FIG. 5). It is obvious that those samples that contain no polyP (“Chitosan control”, “N,O-CMC hydrogel” [Chen X G, Park H J. Chemical characteristics of O-carboxymethyl chitosans related to the preparation conditions. Carbohydrate Polymers 2003; 53:355-359; Luo Y, Teng Z, Wang X, Wang Q. Development of carboxymethyl chitosan hydrogel beads in alcohol-aqueous binary solvent for nutrient delivery applications. Food Hydrocolloids 2013a; 31: 332-339] and N,O-CMC layers, prepared here minus polyP “N O-CMC layer−polyP”) did not change significantly the hemoglobin concentration, as a measure for the free erythrocyte number. However, the N,O-CMC layers, prepared in the present contribution plus polyP “N,O-CMC layer+polyP” and the chitosan polyelectrolyte complex (PEC) containing polyP “Chitosan+polyP” significantly reduced the number of free erythrocytes, and conversely increased the number of erythrocytes bound to the matrices. Comparing the two samples, polyP linked via Ca²⁺ to the N,O-CMC polymer significantly reduced the number of free erythrocytes (0.43±0.07 versus 0.85±0.12), compared to the Chitosan+polyP-PEC sample (Mi F L, Shyu S S, Wong T B, Jang S F, Lee S T, Lu K T. Chitosan-polyelectrolyte complexation for the preparation of gel beads and controlled release of anticancer drug. II. Effect of pH-dependent ionic crosslinking or interpolymer complex using tripolyphosphate or polyphosphate as reagent. J Appl Polymer Sci 1999; 74:1093-1107; Ong S Y, Wu J, Moochhala S M, Tan M H, Lu J. Development of a chitosan-based wound dressing with improved hemostatic and antimicrobial properties. Biomaterials 2008; 29:4323-4332).

The finding of the inventors that polyP linked via Ca²⁺ to the N,O-CMC polymer causes a significantly higher reduction of the erythrocyte number (increase in clotting time) than the published chitosan+polyP-PEC complex (FIG. 5) was unexpected, in particular because it has not been described that the effect of polyP on blood coagulation requires a complex or salt formation of the polyP with calcium ions. The effect of polyP on the clotting time seems to be mediated by a calcium-independent mechanisms (Smith S A, Mutch N J, Baskar D, RohloffP, Docampo R, Morrissey J H (2006) Polyphosphate modulates blood coagulation and fibrinolysis. Proc Natl Acad Sci USA 103:903-908). In addition, it was surprising that polyP, as a calcium complex/salt, is biologically active even after binding to the N,O-CMC polymer.

The following example shows that the inventive method described can also be applied, if polyP linked via Ca²⁺ to the N-carboxymethylated chitosan (N-CMC-polyP) or polyP linked via Ca²⁺ to the O-carboxmethylated chitosan (O-CMC-polyP) instead of N,O-CMC-polyP is used.

The effects of N-CMC-polyP and O-CMC-polyP on mineralization of SaOS-2 cells are summarized in Table 1. Both preparations show a significantly higher effect on mineralization than the published chitosan polyP complex (Mi F L, Shyu S S, Wong T B, Jang S F, Lee S T, Lu K T. Chitosan-polyelectrolyte complexation for the preparation of gel beads and controlled release of anticancer drug. II. Effect of pH-dependent ionic crosslinking or interpolymer complex using tripolyphosphate or polyphosphate as reagent. J Appl Polymer Sci 1999; 74: 1093-1107). However, they were less efficient than N,O-CMC-polyP.

These data surprisingly show that polyP, linked to N,O-CMC or N-CMC or O-CMC via Ca²⁺-linkages has a significantly higher biomineralization potential than when linked otherwise.

Table 1.

Effect of chitosan polyP complex (“Chitosan+polyP”), and N,O-CMC layers minus polyP (“N,O-CMC layer−polyP”), N-CMC layers minus polyP (“N-CMC layer−polyP”), O-CMC layers minus polyP (“O-CMC layer−polyP”), and N,O-CMC layers plus polyP (“N,O-CMC layer+polyP”), N-CMC layers plus polyP (“N-CMC layer+polyP”), O-CMC layers plus polyP (“O-CMC layer+polyP”) on mineralization of SaOS-2 cells. The cells were grown in the absence (control) or presence of the OC (other assays). The extent of biomineralization (Alizarin Red S [AR]) is correlated with the DNA content. The incubation period was 8 days.

| Sample | AR (nmoles/μg DNA) |
|---|---|
| Control | 0.21 ± 0.03 |
| Chitosan + polyP | 0.38 ± 0.04 |
| N,O-CMC layer − polyP | 0.46 ± 0.06 |
| N-CMC layer − polyP | 0.42 ± 0.10 |
| O-CMC layer − polyP | 0.40 ± 0.09 |
| N,O-CMC layer + polyP | 0.93 ± 0.09 |
| N-CMC layer + polyP | 0.80 ± 0.11 |
| O-CMC layer + polyP | 0.77 ± 0.10 |

The effects of N-CMC-polyP and of O-CMC-polyP on the kinetics of blood clotting are summarized in Table 2. Both preparations, like N,O-CMC-polyP, cause a significantly higher effect on blood clotting rates than the published chitosan polyP complex (Mi F L, Shyu S S, Wong T B, Jang S F, Lee S T, Lu K T. Chitosan-polyelectrolyte complexation for the preparation of gel beads and controlled release of anticancer drug. II. Effect of pH-dependent ionic crosslinking or interpolymer complex using tripolyphosphate or polyphosphate as reagent. J Appl Polymer Sci 1999; 74:1093-1107), but they are less effective compared to N,O-CMC-polyP.

Table 2.

Effect of chitosan polyP complex (“Chitosan+polyP”), and N,O-CMC hydrogel (“N,O-CMC hg”), N-CMC hydrogel (“N-CMC hg”), O-CMC hydrogel (“O-CMC hg”), and N,O-CMC layers minus polyP (“N,O-CMC layer−polyP”), N-CMC layers minus polyP (“N-CMC layer−polyP”), O-CMC layers minus polyP (“O-CMC layer−polyP”), and N,O-CMC layers plus polyP (“N,O-CMC layer+polyP”), N-CMC layers plus polyP (“N-CMC layer+polyP”), O-CMC layers plus polyP (“O-CMC layer+polyP”) on blood clotting rates. In the control non-processed chitosan had been added. The absorbance of hemoglobin from lysed noncoagulated erythrocytes was determined.

| Sample | Hemoglobin absorbance (OD₅₄₀) |
|---|---|
| Control | 1.42 ± 0.13 |
| Chitosan + polyP | 0.85 ± 0.12 |
| N,O-CMC hg | 1.29 ± 0.15 |
| N-CMC hg | 1.40 ± 0.21 |
| O-CMC hg | 1.33 ± 0.17 |
| N,O-CMC layer − polyP | 1.47 ± 0.17 |
| N-CMC layer − polyP | 1.51 ± 0.20 |
| O-CMC layer − polyP | 1.41 ± 0.20 |
| N,O-CMC layer + polyP | 0.43 ± 0.07 |
| N-CMC layer + polyP | 0.61 ± 0.11 |
| O-CMC layer + polyP | 0.58 ± 0.08 |

Methods

Polyphosphate

The sodium polyphosphate (Na-polyP of an average chain of 40 phosphate units) used in the Examples has been obtained from Chemische Fabrik Budenheim (Budenheim; Germany).

Preparation of N,O-Carboxymethyl Chitosan

N,O-carboxymethyl chitosan (N,O-CMC) can be prepared from chitosan according to state-of-the-art procedures (Chen X G, Park H J. Chemical characteristics of O-carboxymethyl chitosans related to the preparation conditions. Carbohydrate Polymers 2003; 53:355-359; Chen S C, Wu Y C, Mi F L, Lin Y H, Yu L C, Sung H W. A novel pH-sensitive hydrogel composed of N,O-carboxymethyl chitosan and alginate cross-linked by genipin for protein drug delivery. J Control Release 2004; 96:285-300; Sakairi N, Suzuki S, Ueno K, Han S M, Nishi N, Tokura S. Biosynthesis of hetero-polysaccharides by Acetobacter xylinum-synthesis and characterization of metal-ion adsorptive properties of partially carboxmethylated cellulose. Carbohydrate Polymers 1998; 37:409-414). In brief, 10 g of chitosan (e.g. from shrimp shells, ≥75% (deacetylated) [deacetylated chitin]) is added to water:isopropanol mixture (20 ml: 40 ml) containing 14.1 g NaOH and kept at room temperature for 1 h under mild stirring. After that, 15 g of monochloroacetic acid dissolved in 20 ml of isopropanol is added to the mixture dropwise. The reaction mixture is heated to 50° C. and stirring is continued for 4 h. Then the materials is filtered and washed three times with 80% ethyl alcohol. The resulting solid is dried overnight in an oven at 60° C. to obtain the Na salt of N,O-CMC. For the conversion to the H-form of N,O-CMC, the obtained powder is suspended in 100 ml of aqueous 80% ethyl alcohol solution. Then 10 ml hydrochloric acid (37%) is added and stirred for 30 min. Finally, the suspension is filtrated and washed with ethyl alcohol until a neutral pH is obtained; the material is dried at 60° C. overnight (Sakairi N, Suzuki S, Ueno K, Han S M, Nishi N, Tokura S. Biosynthesis of hetero-polysaccharides by Acetobacter xylinum-synthesis and characterization of metal-ion adsorptive properties of partially carboxmethylated cellulose. Carbohydrate Polymers 1998; 37:409-414). A schematic outline of the reaction is given in FIG. 1A.

Fourier transformed infrared (FTIR) spectroscopy are used in the attenuated total reflectance (ATR) mode to assure the substitutions of carboxymethyl groups at the amino group as well as the primary hydroxyl sites of the chitosan (FTIR-ATR; Varian 660-IR spectrometer with Golden Gate ATR auxiliary) (Chen S C, Wu Y C, Mi F L, Lin Y H, Yu L C, Sung H W. A novel pH-sensitive hydrogel composed of N,O-carboxymethyl chitosan and alginate cross-linked by genipin for protein drug delivery. J Control Release 2004; 96:285-300). Dried powder of sample is placed onto the ATR crystal directly. The spectra are acquired at 4000-750 cm⁻¹ wave numbers with a 4 cm⁻¹ resolution.

Preparation of O-Carboxymethyl Chitosan

O-carboxmethyl chitosan (O-CMC) can be prepared by reacting monochloroacetic acid with chitosan in isopropanol/NaOH solution using state-of-the-art procedures (e.g. Upadhyaya L, Singh J, Agarwal V, Tewari R P. Biomedical applications of carboxymethyl chitosans. Carbohydr Polym 2013; 91:452-466).

Preparation of N-Carboxymethyl Chitosan

N-carboxmethyl chitosan (N-CMC) can be obtained by reacting free amino groups of chitosan with glyoxylic acid and subsequent reduction of the resulting aldimine with sodium borohydride, as described (e.g. Upadhyaya L, Singh J, Agarwal V, Tewari R P. Biomedical applications of carboxymethyl chitosans. Carbohydr Polym 2013; 91:452-466).

Printing of N,O-CMC-Polyphosphate Layers and Tissue-Like-Blocks

N,O-CMC is sterilized, for example, by ultraviolet radiation (254 nm) overnight. Then a solution of 60 mg/ml of N,O-CMC is prepared in physiological saline. After stirring until being homogenous the gel is supplemented with solid Na-polyP until the concentration of 20 mg/ml is reached. The hydrogel preparation formed is completed with 60 mg/ml sodium alginate (e.g., W201502 from Sigma-Aldrich) and stirred at 50° C. until it becomes homogenous. Then this hydrogel is filled into sterile printing cartridges (e.g., 30 ml printing cartridges from Nordson EFD) and centrifuged for 3 min at 1500 rpm to remove remaining air bubbles. After connecting the 0.25 mm tapered polyethylene printing tip (Nordson EFD) the cartridge is placed into the preheated (25° C.) printing head of the 3D-bioplotter; for example, a 3D-Bioplotter, 4th generation blotter, from Envisiontec can be used.

Bioprinting is performed following described procedures (Neufurth M, Wang X H, Schrider H C, Feng Q L, Diehl-Seifert B, Ziebart T, Steffen R, Wang S F and Müller W E G. Engineering a morphogenetically active hydrogel for bioprinting of bioartificial tissue derived from human osteoblast-like SaOS-2 cells. Biomaterials 2014; DOI: 10.1016/j.biomaterials.2014.07.002; in press). The printing solution, composed of 60 mg/ml of N,O-CMC, 60 mg/ml of alginate and 20 mg/ml of Na-polyP is prepared at 25° C. using a pressure of 1.4 bar and a printing speed of 18 mm/s. The pre-flow is set to 0.15 s whereas the post-flow amounts to −0.05 s. Cylindrical scaffolds measuring 50×0.4 mm are designed, sliced and transferred to the printer software as described (Neufurth M, Wang X H, Schröder H C, Feng Q L, Diehl-Seifert B, Ziebart T, Steffen R, Wang S F and Müller W E G. Engineering a morphogenetically active hydrogel for bioprinting of bioartificial tissue derived from human osteoblast-like SaOS-2 cells. Biomaterials 2014; DOI: 10.1016/j.biomaterials.2014.07.002; in press). The strand distance between the printed cylinders is set to 1 mm, resulting in a pore size of the printed layers/blocks of approximately 0.5×0.5 mm. Those scaffolds, layers/blocks, are printed directly into sterile 94 mm Petri dishes, supplemented with 1% [w/v] CaCl₂ as crosslinking solution (Schloßmacher U, Schröder H C, Wang X H, Feng Q, Diehl-Seifert B, Neumann S, Trautwein A, Müller W E G. Alginate/silica composite hydrogel as a potential morphogenetically active scaffold for three-dimensional tissue engineering. RSC Advances 2013; 3:11185-11194). After a ≈2 min incubation period the CaCl₂-solution is drained and the cross-linked scaffolds produced are washed twice with distilled water and once with 70% ethanol. The printing of a two layered scaffold with 5 cm in diameter lasts approximately 6 min. The size of the scaffold samples for the cell culture experiments is 20 mm [diameter]×0.4 mm [thickness].

In the example, a tissue-like block is printed after analysis of the cranial defect, a pig underjaw has been selected, by microtomography [μCT]. The implant dimensions to be printed are predetermined using the computer program Bioplotter RP 2.9 CAD software (Envisiontec). Using the same software, the cylinders are subsequently sliced to individual layers corresponding to the diameter of the printing needle and subsequently transferred to the VisualMachines 3.0.193 printer software (Envisiontec).

Preparation of N,O-CMC Hydrogel Layer and N,O-CMC-polyP-PEC

The N,O-CMC hydrogel is prepared as described (Chen X G, Park H J. Chemical characteristics of O-carboxymethyl chitosans related to the preparation conditions. Carbohydrate Polymers 2003; 53:355-359; Luo Y, Teng Z, Wang X, Wang Q. Development of carboxymethyl chitosan hydrogel beads in alcohol-aqueous binary solvent for nutrient delivery applications. Food Hydrocolloids 2013a; 31: 332-339; Luo Y, Wu C, Lode A, Gelinsky M. Hierarchical mesoporous bioactive glass/alginate composite scaffolds fabricated by three-dimensional plotting for bone tissue engineering. Biofabrication 2013b; 5:015005; doi: 10.1088/1758-5082/5/1/015005). The solid material prepared is layered on the Petri dish (termed “N,O-CMC hydrogel”).

The polyelectrolyte complex (PEC) (Mi F L, Shyu S S, Wong T B, Jang S F, Lee S T, Lu K T. Chitosan-polyelectrolyte complexation for the preparation of gel beads and controlled release of anticancer drug. II. Effect of pH-dependent ionic crosslinking or interpolymer complex using tripolyphosphate or polyphosphate as reagent. J Appl Polymer Sci 1999; 74:1093-107) is prepared from chitosan powder (60 mg/ml) and Na-polyP (20 mg/ml) as described (Ong S Y, Wu J, Moochhala S M, Tan M H, Lu J. Development of a chitosan-based wound dressing with improved hemostatic and antimicrobial properties. Biomaterials 2008; 29:4323-4332). The sample is termed “Chitosan-polyP”.

Scanning Electron Microscopy and Energy-Dispersive X-Ray Spectroscopy

The scanning electron microscope (SEM; HITACHI SU 8000) is coupled to an XFlash 5010 detector, an X-ray detector that allows simultaneous energy-dispersive X-ray (EDX)-based elemental analyses. This is coupled at voltage of 4 kV to the XFlash 5010 detector that is used for element analysis. HyperMap databases are collected, as described (Salge T, Terborg R. EDS microanalysis with the silicon drift detector (CDD): innovative analysis options for mineralogical and material science application. Anadolu Univ J Sci Technol 2009; 10:45-55).

Determination of the Hardness of the N,O-CMC-polyP Scaffold

The hardness of the scaffolds can be determined, for example, by a ferruled optical fiber-based nanoindenter as described (Chavan D, Andres D, Iannuzzi D. Note: ferrule-top atomic force microscope. II. Imaging in tapping mode and at low temperature. Rev Sci Instrum. April 2011; 82(4):046107; doi: 10.1063/1.3579496; Chavan D, van de Watering T C, Gruca G, Rector J H, Heeck K, Slaman M, Iannuzzi D. Ferrule-top nanoindenter: an optomechanical fiber sensor for nanoindentation. Rev Sci Instrum 2012; 83:115110; doi: 10.1063/1.4766959). The indents are depth controlled (10 &m) and the loading and unloading period is set to 2 s. Based on the load-displacement curves the reduced Young's modulus [RedYM] is calculated.

Light Microscopic Analyses

Digital light microscopic studies can be performed, for example, using a VHX-600 Digital Microscope (Keyence) equipped with a VH-Z25 zoom lens.

Mineralization by Cells In Vitro on Chitosan Matrices

For example, human osteogenic sarcoma cells, SaOS-2 cells can be used. The cells are cultivated in McCoy's medium in a humidified incubator at 37° C. and 5% CO₂ (Wiens M, Wang X H, Schröder H C, Kolb U, Schloßmacher U, Ushijima H, Müller W E G. The role of biosilica in the osteoprotegerin/RANKL ratio in human osteoblast-like cells. Biomaterials 2010a; 31:7716-7725). Culture medium/fetal calf serum [FCS] is changed every 3 d. Where mentioned the cells are exposed to the osteogenic cocktail [OC], containing 10 nM dexamethasone, 5 mM β-glycerophosphate and 50 mM ascorbic acid. The scaffold samples 20 mm [diameter]×0.4 mm [thickness] are placed to the bottom of the 24-well pates.

The extent of mineralization can be assayed, for example, by Alizarin Red S and measured spectrophotometrically (Wiens M, Wang X H, Schloßmacher U, Lieberwirth I, Glasser G, Ushijima H, et al. Osteogenic potential of bio-silica on human osteoblast-like (SaOS-2) cells. Calcif Tissue Intern 2010b; 87:513-524). Prior to the measurement the chitosan matrices are removed from the 40-well plates. The amount of bound Alizarin Red S is expressed in nmoles and correlated to total DNA in the samples.

Effect of Blood Clotting Time

The influence of the N,O-CMC matrices, with and without polyP, on blood clotting time can be determined, for example, by the assay described by (Shih M F, Shau M D, Chang M Y, Chiou S K, Chang J K, Cherng J Y. Platelet adsorption and hemolytic properties of liquid crystal/composite polymers. Int J Pharm 2006; 327:117-125). The samples (100 to 150 mg) are submersed in bottles placed in a thermostated water bath at 37° C. for 10 min. Then 300 μl of human blood sample (acid-citrate-dextrose with 20 l/ml of 100 mM CaCl₂) is dropped on the surface of the matrices until they are completely covered. Then the assays are continued to be incubated (37° C.) for 10 min. Then 15 ml of distilled water are added without disturbing the clotted blood. Subsequently 10 ml aliquots are taken, centrifuged (100×g; 30 s) and the supernatant is collected and the clotting test is performed spectrophotometrically at 542 nm.

Statistical Analysis

The results can be statistically evaluated using paired Student's t-test.

1. A method for the preparation of a scaffold for tissue engineering and repair, comprising the steps of:
i) combining carboxymethyl chitosan, polyphosphate, and alginate; to form a hydrogel;
ii) three-dimensional (3D) printing of the resulting hydrogel; and
iii) hardening, by exposure to calcium ions, of the material after printing.

2. The method according to claim 1, wherein said carboxymethyl chitosan has been formed by carboxymethylation of the amino groups of chitosan (N-carboxymethyl chitosan) or the hydroxy groups of chitosan (O-carboxymethyl chitosan), or both (N,O-carboxymethyl chitosan).

3. The method according to claim 1, wherein non-carboxymethylated (free) amino groups or hydroxy groups, or both, of said carboxymethyl chitosan are acetylated or partially acetylated.

4. The method according to claim 1, wherein said polyphosphate and the alginate are present as a sodium salt.

5. The method according to claim 1, wherein the average chain length of the polyphosphate is between 10 and 100 phosphate units.

6. The method according to claim 5, wherein the average chain length of the polyphosphate is about 40 phosphate units.

7. The method according to claim 1, wherein said alginate has been supplemented with gelatin or another collagen-derived product.

8. The method according to claim 1, wherein said hydrogel as formed is supplemented with an additional morphogenetically active oligomer or polymer.

9. The method according to claim 8, wherein said additional morphogenetically active polymer is polymeric silicic acid (silica) or one of its salts.

10. The method according to claim 9, wherein said polymeric silicic acid has been formed by an enzyme or protein involved in biosilica.

11. The method according to claim 10, wherein a silicatein or a silicatein fusion protein or combinations thereof, as well as a suitable substrate, are present.

12. The method according to claim 11, wherein said silicatein polypeptide or silicatein fusion protein has been produced using a prokaryotic or eukaryotic expression system, or has been produced synthetically.

13. The method according to claim 1, wherein said hydrogel is supplemented with bioactive glass (nano)particles composed of SiO₂:CaO:P₂O₅ or SiO₂:Na₂O:CaO:P₂O₅.

14. The method according to claim 1, wherein said hydrogel is simultaneously printed with a suspension of bioactive glass (nano)particles using a three-dimensional (3D) printing technique.

15. The method according to claim 1, wherein cells are suspended in said hydrogel and the resulting cell-containing hydrogel is subjected to 3D printing and subsequent hardening by exposure to calcium ions, and wherein said cells are not human embryonic stem cells.

16. The method according to claim 15, wherein said cells are bone-forming cells or bone-dissolving cells or their precursors, or a mixture of both.

17. A 3D-bioprinted scaffold, obtained by a method according to claim 1.

18. The 3D-bioprinted scaffold according to claim 17, wherein said scaffold is in the form of bone implant material or a part of such material.

19. The 3D-bioprinted scaffold according to claim 17, wherein said scaffold is in the form of a customized implant fabricated by 3D printing, 3D cell printing, or another rapid prototyping/solid free-form fabrication process.

20. A method for treatment of bone defects wherein said method utilizes the bone implant material according to claim 18.
